(19) United States
(12) Patent Application Publication
(10) Pub. No.: US 2018/0021247 A1
(43) Pub. Date: Jan. 25, 2018

(54) TOPICAL COMPOSITIONS COMPRISING HYDROXY ACIDS AND CANNABINOIDS FOR SKIN CARE

(71) Applicant: Kannalnnovations LLC, New York, NY (US)

(72) Inventors: Babek Ghalili, New York, NY (US); Kevin McGovern, Palm Beach, FL (US)

(73) Assignee: Kannalnnovations LLC, New York, NY (US)

(21) Appl. No.: 15/609,253

(22) Filed: May 31, 2017

Related U.S. Application Data
(63) Continuation of application No. 15/233,251, filed on Aug. 10, 2016, now abandoned.
(60) Provisional application No. 62/203,698, filed on Aug. 11, 2015.

(51) Int. Cl. A61K 8/97; A61K 47/44; A61K 31/194; A61K 31/19; A61K 36/185; A61K 45/06; A61K 9/00; A61K 8/92; A61K 8/73; A61K 8/365; A61Q 19/08; A61K 8/34; A61K 31/192; A61K 31/366; A61K 47/12

(52) U.S. Cl. CPC A61K 8/97; A61K 31/366; A61K 47/12; A61K 31/194; A61K 31/19; A61K 36/185; A61K 45/06; A61K 9/0014; A61K 8/922; A61K 8/731; A61K 8/365; A61Q 19/08; A61K 8/345; A61K 31/192; A61K 47/44; A61Q 17/04

(57) ABSTRACT

The present invention relates to compositions and methods for the prevention and treatment of skin disorders and for the rejuvenation of the skin. In particular, the application describes topical compositions and methods of treatments comprising the combined use of one or more cannabinoids and one or more hydroxy acids in a suitable carrier.

### CROSS-REFERENCE TO RELATED APPLICATIONS

This application claims the benefit of priority from U.S. Provisional Application No. 62/203,698, filed Aug. 11, 2015, the content of which is incorporated by reference in its entirety.

### FIELD OF THE INVENTION

The present invention relates to the use of hydroxy acids and cannabinoids for skin care.

### BACKGROUND OF THE INVENTION

The skin is the largest organ of the body, with a surface area of 18 square feet. In the epidermis, the keratinocytes produce keratin, a protein that gives skin its strength and flexibility and waterproofs the skin surface. Collagen and elastic fibers in the dermis give strength to the skin. The skin is continuously exposed to changes in the external environment, including oxidative insults, heat, cold, UV radiation, injury, and mechanical stresses. The stratum corneum, composed of terminally differentiated keratinocytes, constitutes the natural barrier that prevents loss of water and penetration of infectious agents, such as bacteria and viruses, and foreign particles. Keratin intermediate filaments provide the cells with mechanical resilience and protects them against physical stress. Disruption of the keratin scaffold leads to tissue and cell fragility in the skin and its appendages (hair, nail, glands), oral mucosa, and cornea, and exposes the skin to pathological conditions and diseases.

Dermatitis, also known as eczema, is an inflammation of the skin that is characterized by the presence of itchy, erythematous, vesicular, weeping, and crusting patches. Inflammatory agents include bacteria, fungi, viruses, and autoimmune, allergic, hormonal and malignant inflammatory agents. The most common skin diseases or disorders include eczema, psoriasis, dermatitis, itching dermatosis, rosacea, perioral dermatitis, acne, non-melanoma skin cancer and melanoma. Although symptoms vary, recurrent dermatitis conditions include pruritus, dryness and skin rashes, which may be accompanied by redness, skin swelling, itching and dryness, crusting, flaking, blistering, cracking, oozing, or bleeding. Common forms of dermatitis include atopic dermatitis, an allergic disease characterized by the presence of itchy rashes, contact dermatitis, which may be caused by an allergen or an irritant, xerotic eczema, which is caused by dry skin, and seborrheic dermatitis, which is more common in infants.

Sunlight is a major cause of skin aging. Symptoms of skin aging are wrinkles, age spots and dryness. While treatment with moisturizers and steroid creams may temporarily control skin disorder and aging symptoms by reducing inflammation and smoothing wrinkles, the relief is only temporary. There is no known cure for dermatitis, and systemic side effects prevent long term treatment of chronic dermatological conditions with steroids.

Accordingly, there is a need in the art for improved treatment options for improving skin condition, delaying and reducing the effects of skin aging and treating or preventing skin disorders.

### SUMMARY OF THE INVENTION

It is, therefore, an object of the invention to provide solutions to the aforementioned problems, among other objects.

One embodiment of the invention is a topical composition for treating skin that comprises a therapeutically effective amount of at least one cannabinoid and a therapeutically effective amount of a hydroxy acid in a topically acceptable carrier. The at least one cannabinoid and at least one hydroxy acid may optionally be the only active ingredients of the composition. In one aspect of the invention, the cannabinoids are present in the topical composition in a concentration between 0.1 and 30% by weight of the composition. Preferably, the cannabinoids are one or more of a natural phytocannabinoid, an organic cannabinoid, an endocannabinoid, a cannabinoid analog, a cannabinoid derivative, a synthetic cannabinoid and a cannabinoid receptor agonist. The hydroxy acid is an alpha hydroxy acid, a beta hydroxy acid or a combination thereof. In one aspect of the invention, the hydroxy acid is an alpha hydroxy acid, and the alpha hydroxy acid is lactic acid, citric acid, glycolic acid, mandelic acid, benzylic acid, malic acid, tartaric acid, gluconolactone, galactonolactone, glucuronolactone, galacturonolactone, gulonolactone, ribonolactone, saccharic acid lactone, pantoyllactone, glucoheptonolactone, mannonolactone, or galactoheptonolactone. In a different aspect of the invention, the hydroxy acid is a beta hydroxy acid, and the beta hydroxy acid is salicylic acid or lipohydroxy acid.

In one aspect of the invention, the cannabinoid is hemp oil or human breast milk.

In a different aspect of the invention, the cannabinoid is one or more of cannabigerolic acid (CBGA), cannabigerolic acid monomethylether (CBGAM), cannabigerol (CBG), cannabigerol monomethylether (CBGM), cannabigerovarinic acid (CBGVA), cannabigerovarin (CBGV), cannabichromenic acid (CBCA), cannabichromene (CBC), cannabichromevarinic acid (CBCVA), cannabichromevarin (CBCV), cannabidiolic acid (CBDA), cannabidiol (CBD), cannabidiol monomethylether (CBDM), cannabidiol-C₄ (CBD-C₄), cannabidivarinic acid (CBDVA), cannabidivarin (CBDV), cannabidiorcol (CBD-C₁), delta-9-tetrahydrocannabinolic acid A (THCA-A), delta-9-tetrahydrocannabinolic acid B (THCA-B), delta-9-tetrahydrocannabinol (THC), delta-9-tetrahydrocannabinolic acid-C₄ (THCA-C₄), delta-9-tetrahydrocannabinol-C₄ (THC-C₄), delta-9-tetrahydrocannabivarinic acid (THCVA), delta-9-tetrahydrocannabivarin (THCV), delta-9-tetrahydrocannabiorcolic acid (THCA-C₁), delta-9-tetrahydrocannabiorcol (THC-C₁), delta-7-cis-iso-tetrahydrocannabivarin, delta-8-tetrahydrocannabinolic acid (Δ⁸-THCA), delta-8-tetrahydrocannabinol (Δ⁸-THC), cannabicyclolic acid (CBLA), cannabicyclol (CBL), cannabicyclovarin (CBLV), cannnabielsoic acid A (CBEA-A), cannabielsoic acid B (CBEA-B), cannabielsoin (CBE), cannabinolic acid (CBNA), cannabinol (CBN), cannabinol methylether (CBNM), cannabinol-C₄ (CBN-C₄), cannabivarin (CBV), cannabinol-C₂ (CBN-C₂), cannabiorcol (CBN-C₁), cannabinodiol (CBND), cannabinodivarin (CBVD), cannabitriol (CBT), 10-ethoxy-9-hydroxy-delta-6a-tetrahydrocannabinol, 8,9-dihydroxy-delta-6a-tetrahydrocannabinol, cannabitriolvarin (CBTV), ethoxy-cannabitriolvarin (CBTVE), dehydrocannabifuran (DCBF), cannabifuran (CBF), cannabichromanon (CBCN), cannabicitran (CBT), 10-oxo-delta-6a-tetrahydrocannabinol (OTHC), delta-9-cis-tetrahydrocannabinol (cis-THC), 3,4,5,6-tetrahydro-7-hydroxy-alpha-alpha-2-trimethyl-9-n-propyl-2,6-methano-2H-1-benzoxocin-5-methanol (OH-iso-HHCV), cannabiripsol (CBR) and trihydroxy-delta-9-tetrahydrocannabinol (triOH-THC).

In yet another aspect of the invention, the cannabinoid is a cannabinoid receptor agonist. Preferably, the cannabinoid receptor agonist comprises one or more of a naphthoylindole, a naphthylmethylindole, a naphthoylpyrrole, a naphthylmethylindene, a phenylacetylindole and a cyclohexylphenol.

In one embodiment of the invention, the hydroxy acid is present in the topical composition in a concentration between 0.1 and 10% by weight of the composition. In one aspect of the invention, the hydroxy acid is an alpha hydroxy acid, wherein the alpha hydroxy acid is lactic acid, citric acid, glycolic acid, mandelic acid, benzylic acid, malic acid, tartaric acid, gluconolactone, galactonolactone, glucuronolactone, galacturonolactone, gulonolactone, ribonolactone, saccharic acid lactone, pantoyllactone, glucoheptonolactone, mannonolactone, or galactoheptonolactone. In a different aspect of the invention, the hydroxy acid is a beta hydroxy acid, and the beta hydroxy acid is salicylic acid or lipohydroxy acid.

In one embodiment, the topical composition may further comprise a stabilizer. Preferably, the stabilizer is selected from the group consisting of guar gum, xanthan gum cellulose hyaluronic acid, polyvinyl pyrrolidone (PVP), alginate, chondritin sulfate, poly gamma glutamic acid, gelatin, chitisin, corn starch and flour, and is present in an amount from about 0.25% to about 30% (w/v).

In a preferred aspect of the invention, the topical composition is in the form of an ointment, a cream, an emulsion, a lotion, a paste, an unguent, a gel or a sunscreen. In yet another preferred aspect, the carrier in the topical composition comprises hemp oil.

In one embodiment, the topical composition further comprises one or more of a thickening agent, an antibiotic, an antiseptic agent, an antifungal, an antibacterial agent, an analgesic or an antiviral agent. In one aspect of the invention, the topical composition may further comprises a UV absorbing agent in an amount between 0.1 and 5% by weight of the composition.

In a different embodiment, the invention provides a method of treating skin, treating a skin disorder, or improving a condition of the skin in a subject in need thereof comprising topically administering to the subject the topical composition of the invention as described above. In one aspect of the invention, the skin disorder is one or more of eczema, psoriasis, dermatitis, itching dermatosis, rosacea, perioral dermatitis, acne, non-melanoma cancer or melanoma. In another aspect of the invention, the subject presents a symptom which is one or more of pruritus, dryness, skin rash, redness, swelling of the skin, itching, crusting, flaking, blistering, cracking, oozing, or bleeding. In yet another aspect, the dermatitis is atopic dermatitis, contact dermatitis, xerotic eczema, or seborrheic dermatitis. Preferably, the composition of the invention is topically administered to the subject in an amount between about 100 nmol to about 1 μmol/cm². In one aspect of the invention, the subject is a mammal. In a preferred aspect of the invention, the mammal is a human.

In yet another embodiment, the invention provides a method for treating or preventing pruritus, dryness of the skin, skin rash, redness, swelling of the skin, itching, crusting, flaking, blistering, cracking, oozing, bleeding or blistering of the skin in a subject in need thereof, that comprises topically administering to the subject the composition of the invention. In one aspect of the invention, the subject has one or more of eczema, psoriasis, dermatitis, itching dermatosis, rosacea, perioral dermatitis, acne, non-melanoma cancer or melanoma. In one embodiment, the dermatitis is atopic dermatitis, contact dermatitis, xerotic eczema, or seborrheic dermatitis. Preferably, the composition of the invention is topically administered to the subject in an amount between about 100 nmol to about 1 μmol/cm². In one aspect of the invention, the subject is a mammal. In a preferred aspect of the invention, the mammal is a human.

The foregoing general description and following brief description of the drawings and the detailed description are exemplary and explanatory and are intended to provide further explanation of the invention as claimed. Other objects, advantages, and novel features will be readily apparent to those skilled in the art from the following detailed description of the invention.

### BRIEF DESCRIPTION OF THE DRAWINGS

FIG. 1 illustrates the chemical structure of some cannabinoids for use according to the invention.

FIG. 2 illustrates the effect of the CBD-AHA day cream composition on skin firmness, as measured by a Cutometer. Measurements were taken on the skin of the subjects on day one prior to the application of the composition, and again after 28 days of daily use of the composition. The results show an average decrease of 1.09% in the R0 parameter, which represents the final distension of skin on the test sites treated with the composition.

FIG. 3 illustrates the effect of the CBD-AHA night cream composition on skin firmness, as measured by a Cutometer. Measurements were taken on the skin of the subjects on day one prior to the application of the composition, and again after 28 days of daily use of the composition. The results show an average decrease of 0.81% in the R0 parameter, which represents the final distension of skin on the test sites treated with the composition.

FIG. 4 illustrates the effect of the CBD-AHA-FS night cream composition on skin firmness, as measured by a Cutometer. Measurements were taken on the skin of the subjects on day one prior to the application of the composition, and again after 28 days of daily use of the composition. The results show an average decrease of 3.37% in the R0 parameter, which represents the final distension of skin on the test sites treated with the composition.

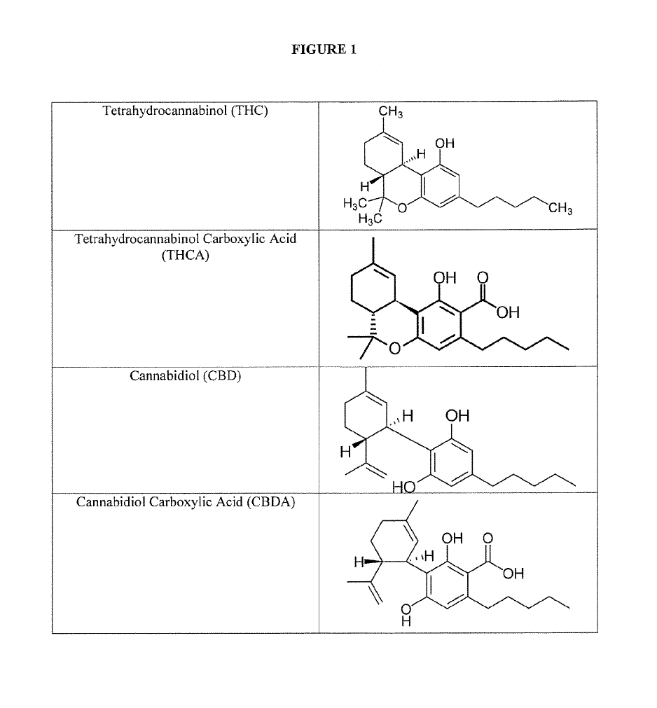

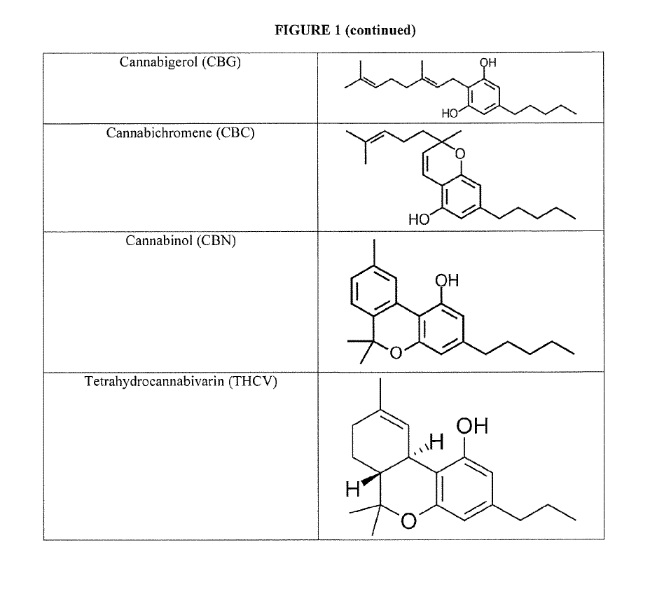

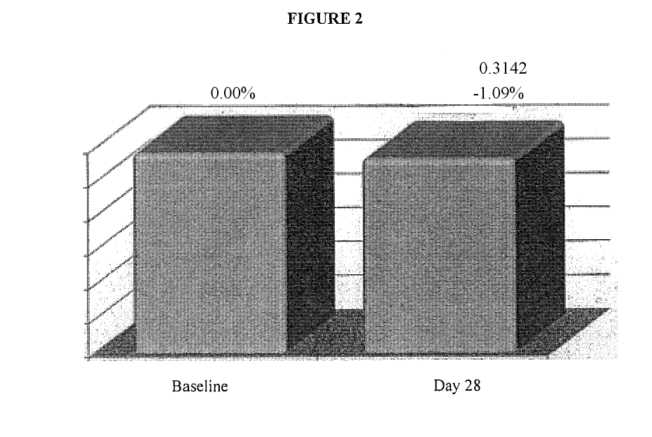

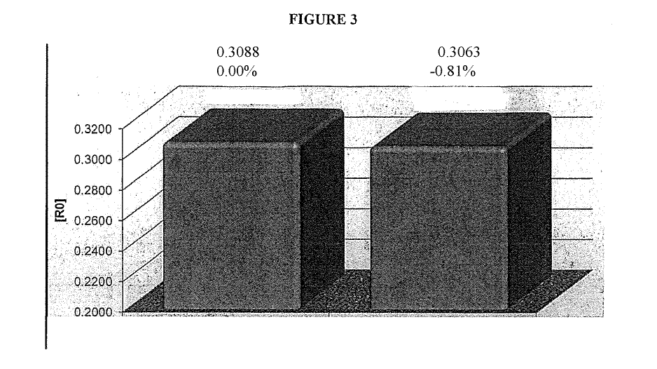

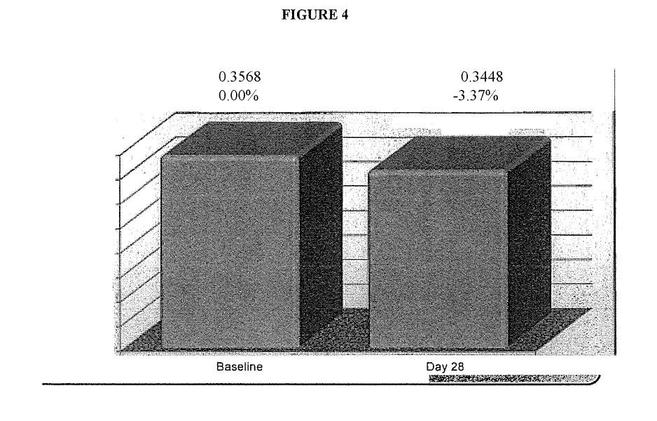

### DETAILED DESCRIPTION OF THE INVENTION

Cannabinoids are terpenophenolic compounds found in Cannabis sativa, an annual plant belonging to the Cannabaceae family. The plant contains more than 400 chemicals and approximately 80 cannabinoids. The latter accumulate mainly in the glandular trichomes. Natural phytocannabinoids occur in the free acid forms within plant tissue. For instance, the psychoactive cannabinoids tetrahydrocannbinol (THC), cannabidiol (CBD), cannabichromene (CBC) and cannabigerol (CBG) exist in their corresponding carboxylic acid forms THCA, CBDA, CBCA and CBGA within plant tissue and are converted to their active forms via non-enzymatic decarboxylation that occurs upon the drying of the plant tissue, or during storage or smoking.

The most active of the naturally occurring cannabinoids is tetrahydrocannabinol (THC), which is used for treating a wide range of medical conditions, including glaucoma, AIDS wasting, neuropathic pain, treatment of spasticity associated with multiple sclerosis, fibromyalgia and chemotherapy-induced nausea. Additionally, THC has been reported to be effective for the treatment of allergies, inflammation, infection, epilepsy, depression, migraine, bipolar disorders, anxiety disorder, drug dependency and drug withdrawal syndromes.

Cannabidiol (CBD), an isomer of THC, is a potent antioxidant and anti-inflammatory compound known to provide protection against acute and chronic neurodegeneration, and relief from chronic pain, inflammation, migraines, arthritis, spasms, epilepsy and schizophrenia.

Cannabigerol (CBG), which is found in high concentrations in hemp, acts as a high affinity α₂-adrenergic receptor agonist, moderate affinity 5-HT_{1A} receptor antagonist and low affinity CB₁ receptor antagonist, and thus may have anti-depressant activity. Cannabichromene (CBC) possesses anti-inflammatory, anti-fungal and anti-viral properties. Tetrahydrocannabivarin (THCV) is known as an appetite suppressant.

The use of cannabinoids in topical compositions is limited by the fact that cannabinoids, because of their hydrophobic nature, must be dissolved in organic solvents that may irritate the skin.

Alpha and beta hydroxy acids are chemical exfoliants. Alpha hydroxy acids are carboxylic acids characterized by the presence of one hydroxyl group attached to the α-position of the carboxyl group, known for their beneficial exfoliating properties and for inducing skin proliferation and new cell growth. Exemplary alpha-hydroxy acids include, but are not limited to, glycolic acid, lactic acid, malic acid, citric acid and tartaric acid. Alpha-hydroxy acids are different from beta-hydroxy acids, such as β-hydroxybutanoic acid, which are carboxylic acids characterized by having one hydroxyl group attached to the β-position of the carboxyl group, as shown in the figure below.

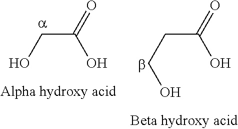 [SMILES: O=C(O)CO ; O=C(O)CCO]

These differences in structure are reflected in differences in solubility, as alpha-hydroxy acids are water-soluble, whereas beta-hydroxy acids are lipid-soluble. Both alpha-hydroxy acids and beta-hydroxy acids can penetrate the stratum corneum and act as exfoliants. Small molecule alpha-hydroxy acids, such as glycolic acid and lactic acid, are best suited to penetrate dry skin and act as humectants and moisturizers. On the other hand, oil-soluble beta hydroxy acids are best suited to penetrate into pores clogged with oily cells and sebum.

The present inventors have unexpectedly discovered that compositions containing hydroxy acids in combination with one or more cannabinoids provide a number of advantages not found when either active agent is used by itself, including reduced skin irritation and fast healing of any skin condition that is enhanced by inflammation including, but not limited to, acne, aging spots, scar formation, eczema and wrinkles. Without being bound to any theory, it is believed that the cannabinoids of the inventive compositions modulate the cannabinoid receptors CB₁R and CB₂R located in the skin and involved in the attenuation of pain and contact allergic reaction, and thus stimulate the proliferation, growth and differentiation of keratinocytes in the skin as well as their immune competence and/or tolerance, while neutralizing the irritating effects of the hydroxy acids. Furthermore, it is believed that the combination of the cannabinoids with the hydroxy acids results in an unexpected synergic anti-inflammatory effect due to the anti-inflammatory properties of the cannabinoids, and contributes to the total wellness of the skin. In fact, skin irritation tests using repeated “open patch” applications of the compositions of the invention on the back of 50 subjects for at least three weeks showed no adverse reactions of any kind and no irritation of the skin in any subject. Accordingly, the compositions containing a combination of one or more hydroxy acids and one or more cannabinoids according to the invention provide greater skin improvement effects than the same compositions comprising either a hydroxy acid or a cannabinoid alone.

As used herein, the terms “cannabinoid” and “cannabinoids” include, but are not limited to, natural phytocannabinoids, organic cannabinoids, endocannabinoids, cannabinoid analogs, cannabinoid derivatives, synthetic cannabinoids and cannabinoid receptor agonists.

Examples of organic cannabinoids include, but are not limited to, hemp oil and human breast milk.

Examples of cannabinoids, cannabinoid analogs and cannabinoid derivatives include, but are not limited to, cannabigerolic acid (CBGA), cannabigerolic acid monomethylether (CBGAM), cannabigerol (CBG), cannabigerol monomethylether (CBGM), cannabigerovarinic acid (CBGVA), cannabigerovarin (CBGV), cannabichromenic acid (CBCA), cannabichromene (CBC), cannabichromevarinic acid (CBCVA), cannabichromevarin (CBCV), cannabidiolic acid (CBDA), cannabidiol (CBD), cannabidiol monomethylether (CBDM), cannabidiol-C₄ (CBD-C₄), cannabidivarinic acid (CBDVA), cannabidivarin (CBDV), cannabidiorcol (CBD-C₁), delta-9-tetrahydrocannabinolic acid A (THCA-A), delta-9-tetrahydrocannabinolic acid B (THCA-B), delta-9-tetrahydrocannabinol (THC), delta-9-tetrahydrocannabinolic acid-C₄ (THCA-C₄), delta-9-tetrahydrocannabinol-C₄ (THC-C₄), delta-9-tetrahydrocannabivarinic acid (THCVA), delta-9-tetrahydrocannabivarin (THCV), delta-9-tetrahydrocannabiorcolic acid (THCA-C₁), delta-9-tetrahydrocannabiorcol (THC-C₁), delta-7-cis-iso-tetrahydrocannabivarin, delta-8-tetrahydrocannabinolic acid (Δ⁸-THCA), delta-8-tetrahydrocannabinol (Δ⁸-THC), cannabicyclolic acid (CBLA), cannabicyclol (CBL), cannabicyclovarin (CBLV), cannnabielsoic acid A (CBEA-A), cannabielsoic acid B (CBEA-B), cannabielsoin (CBE), cannabinolic acid (CBNA), cannabinol (CBN), cannabinol methylether (CBNM), cannabinol-C₄ (CBN-C₄), cannabivarin (CBV), cannabinol-C₂ (CBN-C₂), cannabiorcol (CBN-C₁), cannabinodiol (CBND), cannabinodivarin (CBVD), cannabitriol (CBT), 10-ethoxy-9-hydroxy-delta-6a-tetrahydrocannabinol, 8,9-dihydroxy-delta-6a-tetrahydrocannabinol, cannabitriolvarin (CBTV), ethoxy-cannabitriolvarin (CBTVE), dehydrocannabifuran (DCBF), cannabifuran (CBF), cannabichromanon (CBCN), cannabicitran (CBT), 10-oxo-delta-6a-tetrahydrocannabinol (OTHC), delta-9-cis-tetrahydrocannabinol (cis-THC), 3,4,5,6-tetrahydro-7-hydroxy-alpha-alpha-2-trimethyl-9-n-propyl-2,6-methano-2H-1-benzoxocin-5-methanol (OH-iso-HHCV), cannabiripsol (CBR) and trihydroxy-delta-9-tetrahydrocannabinol (triOH-THC).

Cannabinoid receptor agonists as used herein may include natural and synthetic compounds that are structurally related to natural cannabinoids, and natural and synthetic compounds that are not structurally related to natural cannabinoids, all of which interact with at least one of the cannabinoid receptors CB₁R and CB₂R. Cannabinoid receptor agonists that can be used according to the invention exert the same function as natural cannabinoids and share one or more common features with natural cannabinoids. For example, cannabinoid receptor agonists that are not structurally related to natural cannabinoids are lipid soluble and non-polar, consist of 22 to 26 carbon atoms and have a side-chain comprising more than four and up to nine saturated carbon atoms. Non-limiting examples of cannabinoid receptor agonists that are not structurally related to natural cannabinoids include naphthoylindoles, naphthylmethylindoles, naphthoylpyrroles, naphthylmethylindenes, phenylacetylindoles, such as benzoylindoles, and cyclohexylphenol s.

Exemplary cannabinoid receptor agonists include, but are not limited to, compounds of the molecular formula C₂₁H₃₀O₂ represented by the structure:

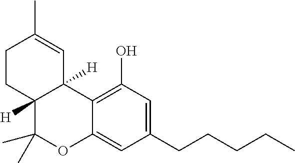 [SMILES: CCCCCc1cc(O)c2c(c1)OC(C)(C)[C@@H]1CCC(C)=C[C@@H]21]

compounds of the molecular formula C₂₅H₃₈O₃ represented by the structure:

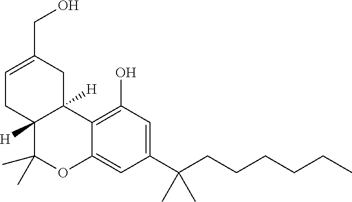 [SMILES: CCCCCCC(C)(C)c1cc(O)c2c(c1)OC(C)(C)[C@@H]1CC=C(CO)C[C@@H]21]

compounds of the molecular formula C₂₁H₃₄O₂ represented by the structure:

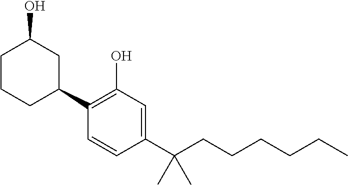 [SMILES: CCCCCCC(C)(C)c1ccc([C@H]2CCC[C@@H](O)C2)c(O)c1]

compounds of the molecular formula C₂₄H₂₃NO represented by the structure:

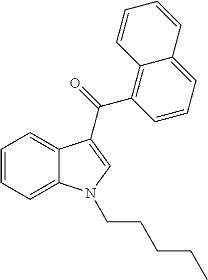 [SMILES: CCCCCn1cc(C(=O)c2cccc3ccccc23)c2ccccc21]

and compounds of the molecular formula C₂₂H₂₅NO₂ represented by the structure:

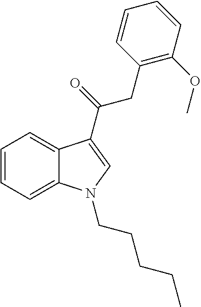 [SMILES: CCCCCn1cc(C(=O)Cc2ccccc2OC)c2ccccc21]

Accordingly, the present invention provides a topical composition and methods for the treatment of a skin disorder or rejuvenation of the skin that comprise administering a topical composition, wherein the topical composition comprises a therapeutically effective amount of at least one cannabinoid and a therapeutically effective amount of at least one hydroxy acid in a pharmaceutically acceptable carrier. Preferably, the carrier is an oil. Even more preferably, the oil is hemp oil. The use of refined hemp oil in the topical compositions and methods of the invention is particularly advantageous, as hemp oil has a high content of antioxidants and cannabinoids having anti-inflammatory effects, such as cannabidiol. Hemp oil is also enriched in omega-6 fatty acids and has a 3:1 ratio of omega-6 to omega-3 essential fatty acids, which matches the balance required by the human body.

In one aspect of the invention, the cannabinoids are present in the topical composition in a concentration between 0.1 and 30% by weight of the composition. Preferably, the cannabinoids are one or more of tetrahydrocannbinol (THC), cannabidiol (CBD), cannabigerol (CBG), cannabichromene (CBC), tetrahydrocannabivarin (THCV), analogs thereof, derivatives thereof, organic and synthetic cannabinoids and cannabinoid receptor agonists as described above. In one aspect of the invention, the cannabinoids in the topical composition comprise one or more of a natural phytocannabinoid, an organic cannabinoid, an endocannabinoid, a cannabinoid analog, a cannabinoid derivative, a synthetic cannabinoid and a cannabinoid receptor agonist. The cannabinoid receptor agonist may comprise one or more of a naphthoylindole, a naphthylmethylindole, a naphthoylpyrrole, a naphthylmethylindene, a phenylacetylindole and a cyclohexylphenol.

As used herein, the term “hydroxy acid” includes, but is not limited to, alpha-hydroxy acid and beta-hydroxy acid. The hydroxy acids are present in the topical composition in a concentration between 0.1 and 10% by weight of the composition. Alpha hydroxy acids that may be used according to the invention comprise, but are not limited to, organic carboxylic acids in which one hydroxyl group is attached to the alpha carbon of the acids. The generic structure of alpha hydroxy acids may be represented by the formula (Ra) (Rb) C (OH) COOH, wherein Ra and Rb are each H, F, Cl, Br, alkyl, aralkyl or aryl group of saturated or unsaturated, isomeric or non-isomeric, straight or branched chain or cyclic form, having 1 to 25 carbon atoms. Ra and Rb may also carry an OH, CHO, COOH or alkoxy group having 1 to 9 carbon atoms. The hydroxy acids may be present in the topical composition as a free acid or in lactone form, or in a salt form with an organic base or an inorganic alkali. The hydroxy adds may also exist as stereoisomers as D, L, and DL forms when Ra and Rb are not identical.

Typical alkyl, aralkyl and aryl groups for Ra and Rb include, but are not limited to, methyl, ethyl, propyl, isopropyl, butyl, pentyl, octyl, lauryl, stearyl, benzyl and phenyl. Alpha hydroxy acids include (1) alkyl alpha hydroxyacids; (2) aralkyl and aryl alpha hydroxyacids; (3) polyhydroxy alpha hydroxyacids; and (4) polycarboxylic alpha hydroxyacids.

Alkyl alpha hydroxy acids include, but are not limited to, 2-hydroxyethanoic acid (glycolic acid, hydroxyacetic acid) (H) (H) c (OH) COOH; 2-hydroxypropanoic acid (lactic acid) (CH₃) (s) C (OH) COOH; 2-methyl 2-hydroxypropanoic acid (methyllactic acid) (CH₃) (CH₃) C (OH) COOH; 2-hydroxybutanoic acid (C₂ H₅) (H) C (OH) COOH, 2-hydroxypentanoic acid (C₃ H₇) (H) C (OH) COOH; 2-hydroxyhexanoic acid (C₄ H₉) (H) C (OH) COOH; 2-hydroxyheptanoic acid (C₅ H₁₁ (H) C (OH) COOH; 2-hydroxyoctanoic acid (C₆ H₁₃) (H) C (OH) COOH; 2-hydroxynonanoic acid (C₇ H₁₅) (H) C (OH) COOH; 2-hydroxydecanoic acid C₈ H₁₇) (H) C (OH) COOH; 2-hydroxyundecanoic acid (C₁₀ H₁₉) (H) C (OH) COOH; 2-hydroxydodecanoic acid (alpha hydroxylauric acid) (C₁₀ H₂₁) (H) C (OH) COOH, 2-hydroxytetradecanoic acid (alpha hydroxymyristic acid) (C₁₂ H₂₅) (H) C (OH) COOH; 2-hydroxyhexadecanoic acid (alpha hydroxypalmitic acid) C₁₄ H₂₉) (H) C (OH) COOH; 2-hydroxyoctadecanoic acid (alpha hydroxystearic acid) (C₁₆ H₁₄) (H) C (OH) COOH; 2-hydroxyeicosanoic acid (alpha hydroxyarachidonic acid) (C₁₈ H₃₇) (H) (OH) COOH.

Aralkyl and aryl alpha hydroxy acids include, but are not limited to, 2-phenyl 2-hydroxyethanoic acid (mandelic acid) (C₆ H₅) (H) C (OH) COOH; 2,2-diphenyl 2-hydroxyethanoic acid (benzylic acid) (C₆ H₅) (C₆ H₅) C (OH) COOH; 3-pphenyl 2-hydroxypropanoic acid (phenyllactic acid) (C₆ H₅ CH₂) (H) C (OH) COOH; 2-pphenyl 2-methyl 2-hydroxyethanoic acid (atrolactic acid) (C₆ H₅) (CH₃) C (OH) COOH; 2-(4′-hydroxyphenyl)2-hydroxyethanoic acid (4-hydroxymandelic acid) (HO—C₆ H₄) (H) C (OH) COOH; 2-(4′-chlorophenyl) 2-hydroxyethanoic acid (4-chloromandelic acid) (Cl—C₆ H₄) (H) C (OH) COOH; 2-(3′-hydroxy-4′-methoxyphenyl) 2-hydroxyethanoic acid (3-hydroxy-4-methoxymandelic acid) (HO—, CH₃ O—C₆ H₃) (H) C (OH) COOH; 2-(4′-hydroxy-3′-methoxyphenyl) 2-hydroxyethanoic acid (4-hydroxy-3-methoxymandelic acid) (HO—, CH₃ O—C₆ H₃) (H) C (OH) COOH; 3-(2′-hydroxyphenyl)2-hydroxypropanoic acid [3-(2′-hydroxyphenyl) lactic acid]HO—C₆ H₄—CH₂ (H) C (OH) COOH; 3-(4′-hydroxyphenyl) 2-hydroxypropanoic acid [3-(4′-hydroxyphenyl) lactic acid]HO—C₆—CH₂ (H) C (OH) COOH; and 2-(3′,4′-dihydroxyphenyl) 2-hydroxyethanoic acid (3,4-dihydroxymandelic acid) HO—, HO—C₆ H₃ (H) C (OH) COOH.

Polyhydroxy alpha hydroxy acids include, but are not limited to, 2,3-dihydroxypropanoic acid (glyceiic acid) (HOCH₂) (H) C (OH) COOH; 2,3,4-trihydroxybutanoic acid (isomers; erythronic acid, threonic acid) HOCH₂ (HO)CH₂ (H) C (OH) COOH; 2,3,4,5-tetrahydroxypentanoic acid (isomers; ribonic acid, arabinoic acid, xylonic acid, lyxonic acid) HOCH₂ (HO) CH₂(HO) CH₂ (H) C (OH) COOH; 2,3,4,5,6-pentahydroxyhexanoic acid (Isomers; allonic acid, altronic acid, gluconic acid, mannoic acid, gulonic acid, idonic acid, galactonic acid, talonic acid) HOCH₂ (HO)CH₂ (HO)CH₂ (HO)CH₂ (H) C (OH) COOH; and 2,3,4,5,6,7-hexahydroxyheptanoic acid (isomers; glucoheptonic acid, galactoheptonic acid etc.) HOCH₂ (HO) CH₂ (HO) CH₂ (HO) CH₂ (HO) CH₂ (H) C (OH) COOH.

Polycarboxylic alpha hydroxy acids include, but are not limited to, 2-hydroxypropane-1,3-dioic acid (tartronic acid) HOOC (H) C (OH) COOH; 2hydroxybutane-1,4-dioic acid (malic acid) HOOC CH₂ (H) C (OH) COOH; 2,3-dihydroxybutane-1,4-dioic acid (tartaric acid) HOOC (HO)CH (Hi) C (OH) COOH; 2-hydroxy-2-carboxypentane-1,5-dioic acid (citric acid) HOOC CH₂ C (OH) (COOH) CH₂ COOH; 2,3,4,5-tetrahydroxyhexane-1,6-dioic acid (isomers; saccharic acid, mucic acid etc.) HOOC (CHOH)₄ COOH.

Lactone forms include, but are not limited to, gluconolactone, galactonolactone, glucuronolactone, galacturonolactone, gulonolactone, ribonolactone, saccharic acid lactone, pantoyllactone, glucoheptonolactone, mannonolactone, and galactoheptonolactone.

In a preferred aspect of the invention, the alpha hydroxy acid is lactic acid, citric acid, glycolic acid, mandelic acid, benzylic acid, malic acid, tartaric acid, gluconolactone, galactonolactone, glucuronolactone, galacturonolactone, gulonolactone, ribonolactone, saccharic acid lactone, pantoyllactone, glucoheptonolactone, mannonolactone, or galactoheptonolactone. In an additional preferred aspect of the invention, the beta-hydroxy acid is salicylic acid.

In one embodiment, the topical composition may further comprise a stabilizer. Preferably, the stabilizer is selected from the group consisting of guar gum, xanthan gum cellulose hyaluronic acid, polyvinyl pyrrolidone (PVP), alginate, chondritin sulfate, poly gamma glutamic acid, gelatin, chitisin, corn starch and flour, and is present in an amount from about 0.25% to about 30% (w/v).

In a preferred aspect of the invention, the topical composition is in the form of an ointment, a cream, an emulsion, a lotion, a paste, an unguent, a gel or a sunscreen. In yet another preferred aspect, the carrier in the topical composition comprises hemp oil.

Creams according to the inventions include water-in-oil or oil-in-water emulsions and may further comprise a cleansing agent, an emollient and an aromatic chemical compound.

Ointments and unguents according to the invention optionally contain oil and water in a ratio from 2:1 to 7:1, and may further comprise a wax, alcohols and petroleum-based mollifying agents.

Gels according to the invention optionally contain a vegetable oil up to 5% by weight of the total composition, water and a thickening agent. Preferably, the thickening agent is a natural polysaccharide, such as xanthan gum, carrageen, an alginate or cellulose gum.

Pastes according to the invention may optionally contain aloe gel and beeswax.

Lotions according to the invention include oil-in-water or water-in-oil emulsions and may comprise cetyl alcohol, an emulsifier, a fragrance, glycerol, petroleum jelly, a dye, one or more preservatives and a stabilizing agent.

A sunscreen composition according to the invention may further comprise a UV absorbing agent in an amount between 0.1 and 5% by weight of the composition. Exemplary UV-absorbing compounds include, but are not limited to, benzone compounds, glyceryl PABA, roxadimate, octocrylene, octyl methoxycinnamate, ethoxyethyl p-methoxycinnamate, homomenthyl salicylate, ethylhexyl salicylate, trolamine salicylate, ecamsule, ensulizole, bemotrizinol and bisoctrizole.

In some embodiments, the topical compositions of the invention may further comprise one or more active agents, such as an antibiotic, an antiseptic agent, an antifungal, an antibacterial agent, an analgesic or an antiviral agent. In additional embodiments, the topical compositions of the invention may further comprise anesthetics, anti-cancer agents, antiacne agents, humectants, such as cationic, ionic and non-ionic surfactant, moisturizers, antipruritic agents, antiperspirants, antipsoriatic agents, antiseborrheic agents, antiaging and anti-wrinkle agents, skin lightening agents, depigmenting agents and vitamins.

Exemplary antibiotics include, but are not limited to, ampicillin, bacampicillin, carbenicillin indanyl, mezlocillin, piperacillin, ticarcillin, amoxicillin-clavulanic acid, ampicillin-sulbactam, benzylpenicillin, cloxacillin, dicloxacillin, methicillin, oxacillin, penicillin G, penicillin V, piperacillin tazobactam, ticarcillin clavulanic acid, nafcillin, procaine penicillin, cefadroxil, cefazolin, cephalexin, cephalothin, cephapirin, cephradine, cefaclor, cefamandol, cefonicid, cefotetan, cefoxitin, cefprozil, ceftmetazole, cefuroxime, loracarbef cefdinir, ceftibuten, cefoperazone, cefixime, cefotaxime, cefpodoxime proxetil, ceftazidime, ceftizoxime, ceftriaxone, cefepime, azithromycin, clarithromycin, clindamycin, dirithromycin, erythromycin, lincomycin, troleandomycin, cinoxacin, ciprofloxacin, enoxacin, gatifloxacin, grepafloxacin, levofloxacin, lomefloxacin, moxifloxacin, nalidixic acid, norfloxacin, ofloxacin, sparfloxacin, trovafloxacin, oxolinic acid, gemifloxacin, perfloxacin, imipenem-cilastatin, meropenem, and aztreonam. In one embodiment, the amount of the antibiotic in the composition is from 0.01 to 5% by weight of the total composition.

Antiseptic compounds include, but are not limited to, iodine, manuka honey, octenidine dihydrochloride, phenol, polyhexanide, sodium chloride, sodium hypochlorite, calcium hypochlorite, sodium bicarbonate, methyl paraben, and sodium dehydroacetate. In one embodiment, the amount of the antiseptic compound in the topical formulation is from 0.01 to 5% by weight of the total composition.

Antifungal agents include, but are not limited to, amphotericin B, candicidin, filipin, hamycin, natamycin, nystatin, rimocidin, bifonazole, butoconazole, clotrimazole, econazole, fenticonazole, isoconazole, ketoconazole, luliconazole, miconazole, omoconazole, oxiconazole, sertaconazole, sulconazole, tioconazole, albaconazole, fluconazole, isavuconazole, itraconazole, posaconazole, ravuconazole, terconazole, voriconazole, abafungin, amorolfin, butenafine, naftifine, terbinafine, anidulafungin, caspofungin, micafungin, benzoic acid, ciclopirox, flucytosine, griseofulvin, haloprogin, tolnaftate, undecylenic acid, crystal violet, and balsam of Peru. In one embodiment, the amount of the antifungal agent in the topical formulation is from 0.01 to 5% by weight of the total composition.

Analgesic agents include, but are not limited to, methyl salicylate, codeine, morphine, methadone, pethidine, buprenorphine, hydromorphine, levorphanol, oxycodone, fentanyl, and a non-steroidal anti-inflammatory drug. The amount of the analgesic agent in the topical formulation is from 0.01 to 5% by weight of the total composition.

Anti-viral agents include, but are not limited to, acyclovir, famciclovir, penciclovir, valacyclovir, trifluridine, docosanol, amantadine, rimantadine, oseltamivir, and zanamivir. The amount of the anti-viral agent in the topical formulation is from 0.01 to 5% by weight of the total composition.

In some embodiments, the topical composition of the invention may further comprise a stabilizer selected from the group consisting of guar gum, xanthan gum cellulose hyaluronic acid, polyvinyl pyrrolidone (PVP), alginate, chondritin sulfate, poly gamma glutamic acid, gelatin, chitisin, corn starch and flour, in an amount from about 0.25% to about 2% (w/v).

In some embodiments, the composition for topical application to the skin comprises a therapeutically effective amount of at least one cannabinoid, a therapeutically effective amount of an alpha hydroxy acid, and hemp oil.

The cannabinoids according to the invention may be obtained as an extract from a cannabis plant for medical use, such as Cannabis sativa and Cannabis indica, by extracting the trichomes of the plants in a solvent and heating the mixture to evaporate the solvent. Examples of extraction technologies that may be used include, but are not limited to, CO₂ extraction and microwave extraction. The cannabinoids according to the invention may also be obtained as an extract from a cannabis transgenic plant that overexpresses one or more particular cannabinoids or that does not express or under-expresses one or more particular cannabinoids. Synthetic cannabinoids may be prepared according to the technologies known to those skilled in the art. In the alternative, endogenous nucleic acid sequences may be extracted from a cannabis plant and used to produce cannabinoids by recombinant technology.

The topical compositions of the invention may be prepared by dissolving the dry extracts of cannabinoids in an oil, preferably hemp oil, and by adding the cannabinoid solution to a composition containing the hydroxy acids. The hydroxy acid composition may be an alcohol solution in which the hydroxy acids are dissolved, or the hydroxy acids may be dissolved in an alcohol-free solution. In an alternative embodiment, the hydroxy acids are dissolved in a composition comprising hemp oil or one or more cannabinoid, analogs thereof, derivatives thereof, organic and synthetic cannabinoids and cannabinoid receptor agonists as described above, wherein the one or more cannabinoids, analogs thereof, derivatives thereof, organic and synthetic cannabinoids and cannabinoid receptor agonists are dissolved in an oil. Preferably, the oil is a vegetable oil. Even more preferably the vegetable oil is hemp oil.

In a different embodiment, the invention provides a method to treat a skin disorder or rejuvenate the skin in a subject in need thereof, that comprises topically administering to the subject the topical composition of the invention as described above. The skin disorder may be one or more of eczema, psoriasis, dermatitis, itching dermatosis, rosacea, perioral dermatitis, acne, non-melanoma cancer or melanoma. The subject may present the first signs of irritation, or presents a severe symptom which is one or more of pruritus, dryness, skin rash, redness, swelling of the skin, itching, crusting, flaking, blistering, cracking, oozing, and bleeding. The dermatitis may be atopic dermatitis, contact dermatitis, xerotic eczema, or seborrheic dermatitis. In a preferred embodiment, the composition of the invention is topically administered to the subject in an amount between about 100 nmol to about 1 μmol/cm². In one aspect of the invention, the subject is a mammal. In a preferred aspect of the invention, the mammal is a human.

In yet another embodiment, the invention provides a method for treating or preventing pruritus, dryness of the skin, skin rash, redness, swelling of the skin, itching, crusting, flaking, blistering, cracking, oozing, bleeding or blistering of the skin in a subject in need thereof, that comprises topically administering to the subject the composition of the invention. The subject may be disease-free or may be suspected of having or have one or more skin conditions, such as eczema, psoriasis, dermatitis, itching dermatosis, rosacea, perioral dermatitis, acne, non-melanoma cancer or melanoma. The dermatitis may be atopic dermatitis, contact dermatitis, xerotic eczema, or seborrheic dermatitis. In a preferred embodiment, the composition of the invention is topically administered to the subject in an amount between about 100 nmol to about 1 μmol/cm². In one aspect of the invention, the subject is a mammal. In a preferred aspect of the invention, the mammal is a human.

The present invention thus generally described, will be understood more readily by reference to the following examples, which are provided by way of illustration only, and are not intended to be limiting the present invention.

### EXAMPLES

### Example 1: Preparation of Topical Compositions Comprising Alpha Hydroxy Acid and Cannabinoids

A. Preparation of Alpha Hydroxy Acid Compositions in Alcohol

5.0 grams of lactic acid, glycolic acid, citric acid, mandelic acid, benzylic acid, malic acid, tartaric acid or gluconolactone are dissolved in propylene glycol. The mixture is shaken and 4.0 grams of hydroxypropylcellulose are slowly added to the mixture to avoid clamping.

B. Preparation of Cannabinoid Compositions

Trichomes and leaves are collected from Cannabis sativa and Cannabis indica, dried in air after harvest, finely ground and dissolved into a solvent in a volume ratio of 1:5. The solution is heated to evaporate the solvent and the dry mixture is dissolved in hemp oil.

C. Preparation of Topical Compositions

The solution of alpha hydroxy acid is added to the hemp oil composition containing the cannabinoids and the mixture is agitated and formulated into compositions for topical administration.

### Example 2: Preparation of Topical Compositions Comprising Alpha Hydroxy Acid and Hemp Oil

5.0 grams of lactic acid, glycolic acid, citric acid, mandelic acid, malic acid, tartaric acid or gluconolactone are dissolved in hemp oil and the solution is formulated into compositions for topical administration.

### Example 3: Treatment of Dermatitis

20 subjects with severe dermatitis are divided into four groups, 5 subjects per group. The subjects are instructed to topically apply a lotion two times daily for two weeks. Group one is treated for two weeks with a lotion containing hydroxy acids. Group two is treated for two weeks with a lotion containing cannabinoids. Group three is treated for two weeks with a lotion containing hydroxy acids and cannabinoids in hemp oil prepared according to Example 1. Group four is treated for two weeks with a lotion containing hydroxy acids and hemp oil prepared according to Example 2. The effects of the different treatments are evaluated after two weeks.

### Example 4: Topical Anti-Aging Compositions

Three different anti-aging compositions, “CBD-AHA day cream,” “CBD-AHA night cream” and “CBD-AHA-FS night cream” were used in this set of experiments. Each composition contained the following ingredients:

CBD-AHA-Day Cream

Ingredients: Aqua (Deionized Water), Aloe Barbadensis Leaf (Aloe Vera Gel) Juice, Glycerin, Cetearyl Olivate, Sorbitan Olivate, Stearic Acid, Cetyl Alcohol, Glycolic Acid, Saccharide Isomerate, Butylene Glycol, Carbomer, Polysorbate 20, Palmitoyl Oligopeptide, Palmitoyl Tetrapeptide-7, Cannabis Sativa (Hemp) Seed Oil, Hesperidin Methyl Chalcone, Steareth-20, Dipeptide-2, Xanthan Gum, Mangifera Indica (Mango) Seed Butter, Butyrospermum Parkii (Shea) Butter, Caprylic/Capric Triglyceride, Simmondsia Chinensis (Jojoba) Seed Oil, Cocos Nucifera (coconut) Oil, Allantoin, Citrullus Vulgaris (Watermelon) Fruit Extract, Camellia Sinensis (Green Tea) Leaf Extract, Punica Granatum (Pomegranate) Extract, Cucumis Sativus (Cucumber) Extract, Glycyrrhiza Glabra (Licorice) Root Extract, Astaxanthin, Cranberry Seed Oil, Thioctic Acid, Hyaluronic Acid, Ascorbic Acid (Vitamin C), Retinyl Palmitate (Vitamin A), Tocopheryl Acetate (Vitamin E), Soluble Collagen, Phenoxyethanol, Ethylhexylglycerin.

CBD-AHA-Night Cream

Ingredients: Aqua (Deionized Water), Aloe Barbadensis Leaf (Aloe Vera Gel) Juice, Cetearyl Olivate, Sorbitan Olivate, Cetyl Alcohol, Glycerin, Stearic Acid, Glycolic Acid, Saccharide Isomerate, Cannabis Sativa (Hemp) Seed Oil, Xanthan Gum, Mangifera Indica (Mango) Seed Butter, Butyrospermym Parkii (Shea) Butter, Caprylic/Capric Triglyceride, Simmondsia Chinensis (Jojoba) Seed Oil, Cocos Nucifera (Coconut) Oil, Allantoin, Citrullus Vulgaris (Watermelon) Fruit Extract, Camellia Sinensis (Green Tea) Leaf Extract, Punica Granatum (Pomegranate) Extract, Cucumis Sativus (Cucumber) Extract, Glycyrrhiza Glabra (Licorice) Root Extract, Astaxanthin, Cranberry Seed Oil, Thioctic Acid, Sodium Hyaluronate, Salicylic Acid, Ascorbic Acid (Vitamin C), Retinyl Palmitate (Vitamin A), Tocopheryl Acetate (Vitamin E), Soluble Collagen, Phenoxyethanol, Ethyhexylglycerin.

CBD-AHA-FS Night Cream

Ingredients: Aqua (Deionized Water), Aloe Barbadensis Leaf (Aloe Vera Gel) Juice, Glycerin, Cetearyl Olivate, Sorbitan Olivate, Cetyl Alcohol, Glycerin, Stearic Acid, Glycolic Acid, Cannabis Sativa (Hemp) Seed Oil, Saccharide Isomerate, Xanthan Gum, Mangifera Indica (Mango) Seed Butter, Butyrospermym Parkii (Shea) Butter, Caprylic/Capric Triglyceride, Simmondsia Chinensis (Jojoba) Seed Oil, Cocos Nucifera (Coconut) Oil, Potassium or Sodium Iodide 8 to 20 g/l, Natural Mono or Oligo and Polysaccharide 120 g/l, Iodine 8 g/l, Natural Proteins 11 g/l, Allantoin, Citrullus Vulgaris (Watermelon) Fruit Extract, Camellia Sinensis (Green Tea) Leaf Extract, Punica Granatum (Pomegranate) Extract, Cucumis Sativus (Cucumber) Extract, Glycyrrhiza Glabra (Licorice) Root Extract, Astaxanthin, Cranberry Seed Oil, Thioctic Acid, Sodium Hyaluronate, Salicylic Acid, Ascorbic Acid (Vitamin C), Retinyl Palmitate (Vitamin A), Tocopheryl Acetate (Vitamin E), Soluble Collagen, Phenoxyethanol, Ethyhexylglycerin.

### Example 5: Efficacy of Topical Anti-Aging Compositions

Evaluation

The efficacy of three different anti-aging compositions, “CBD-AHA day cream,” “CBD-AHA night cream” and “CBD-AHA-FS night cream” was evaluated.

The effectiveness of each composition was assessed in 10 different subjects using a Cutometer SEM 575 to measure firmness, and a NOVA® Dermal Phase Meter to determine the content of retained water in the skin. For each composition, skin firmness measurements via Cutometer were performed on the subjects on day one prior to the application of the composition, and again after 28 days of daily use of the composition. All readings were totaled and reported as average scores. The data thus obtained were quoted as percentage differences from baseline at each time point. Comparison of baseline measurements and post-treatment measurements was analyzed using a two-tailed, paired t-test (p<0.05).

Results

With regard to the CBD-AHA day cream, 90% of the subject reported rejuvenation of the skin and an overall improvement in the skin feel and texture after 28 days of daily application. With regard to the CBD-AHA night cream, 90% of the subject reported an overall improvement in skin's clarity, tone and radiance, and a reduction in lines and wrinkles after 28 days of daily application. With regard to the CBD-AHA-FS night cream, 70% of the subject reported an overall improvement in skin's clarity, tone and radiance, and a reduction in lines and wrinkles after 28 days of daily application. All three compositions were effective in improving the overall condition of the skin in the face area after 28 days of daily use. The results are reproduced below.

CBD-AHA-Day Cream

TABLE 1
Skin Firmness

| Panelist ID No. | Baseline | Day 28 | Individual % Difference |
|---|---|---|---|
| 60 1825 | 0.29 | 0.285 | −1.72% |
| 56 3884 | 0.235 | 0.2375 | 1.06% |
| 62 5219 | 0.2775 | 0.2825 | 1.8% |
| 58 7412 | 0.275 | 0.2729 | −0.78% |
| 39 1287 | 0.35 | 0.34 | −2.86% |
| 54 4408 | 0.2975 | 0.2988 | 0.42% |
| 62 0798 | 0.39 | 0.375 | −3.85% |
| 64 7718 | 0.405 | 0.385 | −4.94% |
| 92 5874 | 0.3638 | 0.3525 | −3.09% |
| 60 7847 | 0.2925 | 0.3125 | 6.84% |
| Mean | 0.3176 | 0.3142 | |

| | |
|---|---|
| % Difference | −1.09% |
| P | 0.366 |
| T | 0.926 |

The skin firmness data presented in Table 1 show a decrease of up to 4.94% in the R0 parameter, which represented the final distension of skin on the test site. These results indicated tightness and elasticity on the test sites treated with the CBD-AHA day cream.

CBD-AHA-Night Cream

TABLE 2
Skin Firmness

| Panelist ID No. | Baseline | Day 28 | Individual % Difference |
|---|---|---|---|
| 46 2263 | 0.345 | 0.385 | 11.59% |
| 38 3147 | 0.395 | 0.385 | −2.53% |
| 64 1074 | 0.3125 | 0.3025 | −3.2% |
| 56 7836 | 0.305 | 0.2675 | −12.3% |
| 44 5227 | 0.305 | 0.2575 | −15.57% |
| 46 9007 | 0.26 | 0.255 | −1.92% |
| 78 1833 | 0.32 | 0.3475 | 8.59% |
| 68 3246 | 0.27 | 0.3025 | 12.04% |
| 72 3479 | 0.3175 | 0.3 | −5.51% |
| 46 7249 | 0.2575 | 0.26 | 0.97% |
| Mean | 0.3088 | 0.3063 | |

| | |
|---|---|
| % Difference | −0.81% |
| P | 0.791 |
| T | 0.269 |

The skin firmness data presented in Table 2 show a decrease of up to 15.57% in the R0 parameter, which represented the final distension of skin on the test site. These results indicated tightness and elasticity on the test sites treated with the CBD-AHA night cream.

CBD-AHA-FS Night Cream

TABLE 3
Skin Firmness

| Panelist ID No. | Baseline | Day 28 | Individual % Difference |
|---|---|---|---|
| 62 2435 | 0.3725 | 0.355 | −4.7% |
| 62 6204 | 0.3515 | 0.3425 | −2.56% |
| 62 7072 | 0.395 | 0.37 | −6.33% |
| 54 1210 | 0.36 | 0.325 | −9.72% |
| 60 8470 | 0.375 | 0.3575 | −4.67% |
| 60 5848 | 0.3137 | 0.3325 | 5.99% |
| 50 2032 | 0.3475 | 0.3125 | −10.07% |
| 62 3438 | 0.325 | 0.3175 | −2.31% |
| 68 5565 | 0.4075 | 0.405 | −0.61% |
| 58 4837 | 0.32 | 0.33 | 3.13% |
| Mean | 0.3568 | 0.3448 | |

| | |
|---|---|
| % Difference | −3.37% |
| P | 0.061 |
| T | 1.991 |

The skin firmness data presented in Table 3 show a decrease of up to 10.07% in the R0 parameter, which represented the final distension of skin on the test site. These results indicated tightness and elasticity on the test sites treated with the CBD-AHA-FS night cream.

### Example 6: Evaluation of Skin Irritation or Sensitization by the Topical Anti-Aging Compositions

A Repeat Insult Patch Test (RIPT) was used to assess the irritation and sensitization potential of two different anti-aging compositions, the “CBD-AHA day cream,” and the “CBD-AHA-FS night cream” as described above.

The effect of each composition was assessed in 50 different subjects by applying nine “open patch” applications on the subjects' back each week for three consecutive weeks. In the event of an adverse reaction, the area of erythema and edema would be measured, and subjects would be given 10 to 14 days of rest before the next application. The edema would be estimated by evaluating the skin with respect to the contour of the unaffected normal skin. Each reaction was scored just before application two through nine.

Results

None of the subjects showed any adverse reactions to the application of the two anti-aging compositions, the “CBD-AHA day cream,” and the “CBD-AHA-FS night cream” (data not shown). These results indicate that the compositions of the invention are safe for topical application to human skin and do not cause skin irritation or sensitization.

What is claimed is:

1. A topical composition for the rejuvenation or treatment of skin in the form of an ointment, a cream, an emulsion, a lotion, a paste, an unguent, a gel or a sunscreen comprising therapeutically effective amounts of at least one cannabinoid and at least one hydroxy acid in a topically acceptable carrier, wherein the cannabinoid is present in a concentration between 0.1 and 30% by weight of the composition; wherein the hydroxy acid is present in a concentration between 0.1 and 10% by weight of the composition; and wherein the cannabinoid is one or more of a natural phytocannabinoid, an organic cannabinoid, an endocannabinoid, a cannabinoid analog, a cannabinoid derivative, a synthetic cannabinoid and a cannabinoid receptor agonist.

2. The topical composition of claim 1, wherein the organic cannabinoid is hemp oil or human breast milk.

3. The topical composition of claim 1, wherein the cannabinoid is one or more of cannabigerolic acid (CBGA), cannabigerolic acid monomethylether (CBGAM), cannabigerol (CBG), cannabigerol monomethylether (CBGM), cannabigerovarinic acid (CBGVA), cannabigerovarin (CBGV), cannabichromenic acid (CBCA), cannabichromene (CBC), cannabichromevarinic acid (CBCVA), cannabichromevarin (CBCV), cannabidiolic acid (CBDA), cannabidiol (CBD), cannabidiol monomethylether (CBDM), cannabidiol-C₄ (CBD-C₄), cannabidivarinic acid (CBDVA), cannabidivarin (CBDV), cannabidiorcol (CBD-C₁), delta-9-tetrahydrocannabinolic acid A (THCA-A), delta-9-tetrahydrocannabinolic acid B (THCA-B), delta-9-tetrahydrocannabinol (THC), delta-9-tetrahydrocannabinolic acid-C₄ (THCA-C₄), delta-9-tetrahydrocannabinol-C₄ (THC-C₄), delta-9-tetrahydrocannabivarinic acid (THCVA), delta-9-tetrahydrocannabivarin (THCV), delta-9-tetrahydrocannabiorcolic acid (THCA-C₁), delta-9-tetrahydrocannabiorcol (THC-C₁), delta-7-cis-iso-tetrahydrocannabivarin, delta-8-tetrahydrocannabinolic acid (Δ⁸-THCA), delta-8-tetrahydrocannabinol (Δ⁸-THC), cannabicyclolic acid (CBLA), cannabicyclol (CBL), cannabicyclovarin (CBLV), cannnabielsoic acid A (CBEA-A), cannabielsoic acid B (CBEA-B), cannabielsoin (CBE), cannabinolic acid (CBNA), cannabinol (CBN), cannabinol methylether (CBNM), cannabinol-C₄ (CBN-C₄), cannabivarin (CBV), cannabinol-C₂ (CBN-C₂), cannabiorcol (CBN-C₁), cannabinodiol (CBND), cannabinodivarin (CBVD), cannabitriol (CBT), 10-ethoxy-9-hydroxy-delta-6a-tetrahydrocannabinol, 8,9-dihydroxy-delta-6a-tetrahydrocannabinol, cannabitriolvarin (CBTV), ethoxy-cannabitriolvarin (CBTVE), dehydrocannabifuran (DCBF), cannabifuran (CBF), cannabichromanon (CBCN), cannabicitran (CBT), 10-oxo-delta-6a-tetrahydrocannabinol (OTHC), delta-9-cis-tetrahydrocannabinol (cis-THC), 3,4,5,6-tetrahydro-7-hydroxy-alpha-alpha-2-trimethyl-9-n-propyl-2,6-methano-2H-1-benzoxocin-5-methanol (OH-iso-HHCV), cannabiripsol (CBR) and trihydroxy-delta-9-tetrahydrocannabinol (triOH-THC).

4. The topical composition of claim 1, wherein the cannabinoid receptor agonist comprises one or more of a naphthoylindole, a naphthylmethylindole, a naphthoylpyrrole, a naphthylmethylindene, a phenylacetylindole and a cyclohexylphenol.

5. The topical composition of claim 1, wherein the hydroxy acid is an alpha hydroxy acid, a beta hydroxy acid or a combination thereof.

6. The topical composition of claim 5, wherein the alpha hydroxy acid is lactic acid, citric acid, glycolic acid, mandelic acid, benzylic acid, malic acid, tartaric acid, gluconolactone, galactonolactone, glucuronolactone, galacturonolactone, gulonolactone, ribonolactone, saccharic acid lactone, pantoyllactone, glucoheptonolactone, mannonolactone, or galactoheptonolactone.

7. The topical composition of claim 5, wherein the beta hydroxy acid is salicylic acid or lipohydroxy acid.

8. The topical composition of claim 1, further comprising a stabilizer selected from the group consisting of guar gum, xanthan gum cellulose hyaluronic acid, polyvinyl pyrrolidone (PVP), alginate, chondritin sulfate, poly gamma glutamic acid, gelatin, chitisin, corn starch and flour, in an amount from about 0.25% to about 30% (w/v).

9. The topical composition of claim 1, wherein the carrier comprises hemp oil and wherein the topical composition further comprises one or more of a thickening agent, an antibiotic, an antiseptic agent, an antifungal, an antibacterial agent, an analgesic, an antiviral agent or a UV absorbing agent in an amount between 0.1 and 5% by weight of the composition.

10. A method to treat a skin disorder or rejuvenate the skin in a subject in need thereof comprising topically administering to the subject a composition in the form of an ointment, a cream, an emulsion, a lotion, a paste, an unguent, a gel or a sunscreen comprising therapeutically effective amounts of at least one cannabinoid and at least one hydroxy acid in a pharmaceutically acceptable carrier, wherein the cannabinoid is present in a concentration between 0.1 and 30% by weight of the composition; wherein the hydroxy acid is present in a concentration between 0.1 and 10% by weight of the composition; and wherein the cannabinoid is one or more of a natural phytocannabinoid, an organic cannabinoid, an endocannabinoid, a cannabinoid analog, a cannabinoid derivative, a synthetic cannabinoid and a cannabinoid receptor agonist.

11. The method of claim 10, wherein the skin disorder is one or more of eczema, psoriasis, dermatitis, itching dermatosis, rosacea, perioral dermatitis, acne, non-melanoma cancer or melanoma.

12. The method of claim 10, wherein the subject presents a symptom which is one or more of pruritus, dryness, skin rash, redness, swelling of the skin, itching, crusting, flaking, blistering, cracking, oozing, or bleeding of the skin.

13. The method of claim 11, wherein the dermatitis is atopic dermatitis, contact dermatitis, xerotic eczema, or seborrheic dermatitis.

14. The method of claim 10, wherein the organic cannabinoid is hemp oil or human breast milk.

15. The method of claim 10, wherein the cannabinoid is one or more of cannabigerolic acid (CBGA), cannabigerolic acid monomethylether (CBGAM), cannabigerol (CBG), cannabigerol monomethylether (CBGM), cannabigerovarinic acid (CBGVA), cannabigerovarin (CBGV), cannabichromenic acid (CBCA), cannabichromene (CBC), cannabichromevarinic acid (CBCVA), cannabichromevarin (CBCV), cannabidiolic acid (CBDA), cannabidiol (CBD), cannabidiol monomethylether (CBDM), cannabidiol-C₄ (CBD-C₄), cannabidivarinic acid (CBDVA), cannabidivarin (CBDV), cannabidiorcol (CBD-C₁), delta-9-tetrahydrocannabinolic acid A (THCA-A), delta-9-tetrahydrocannabinolic acid B (THCA-B), delta-9-tetrahydrocannabinol (THC), delta-9-tetrahydrocannabinolic acid-C₄ (THCA-C₄), delta-9-tetrahydrocannabinol-C₄ (THC-C₄), delta-9-tetrahydrocannabivarinic acid (THCVA), delta-9-tetrahydrocannabivarin (THCV), delta-9-tetrahydrocannabiorcolic acid (THCA-C₁), delta-9-tetrahydrocannabiorcol (THC-C₁), delta-7-cis-iso-tetrahydrocannabivarin, delta-8-tetrahydrocannabinolic acid (Δ⁸-THCA), delta-8-tetrahydrocannabinol (Δ⁸-THC), cannabicyclolic acid (CBLA), cannabicyclol (CBL), cannabicyclovarin (CBLV), cannnabielsoic acid A (CBEA-A), cannabielsoic acid B (CBEA-B), cannabielsoin (CBE), cannabinolic acid (CBNA), cannabinol (CBN), cannabinol methylether (CBNM), cannabinol-C₄ (CBN-C₄), cannabivarin (CBV), cannabinol-C₂ (CBN-C₂), cannabiorcol (CBN-C₁), cannabinodiol (CBND), cannabinodivarin (CBVD), cannabitriol (CBT), 10-ethoxy-9-hydroxy-delta-6a-tetrahydrocannabinol, 8,9-dihydroxy-delta-6a-tetrahydrocannabinol, cannabitriolvarin (CBTV), ethoxy-cannabitriolvarin (CBTVE), dehydrocannabifuran (DCBF), cannabifuran (CBF), cannabichromanon (CBCN), cannabicitran (CBT), 10-oxo-delta-6a-tetrahydrocannabinol (OTHC), delta-9-cis-tetrahydrocannabinol (cis-THC), 3,4,5,6-tetrahydro-7-hydroxy-alpha-alpha-2-trimethyl-9-n-propyl-2,6-methano-2H-1-benzoxocin-5-methanol (OH-iso-HHCV), cannabiripsol (CBR) and trihydroxy-delta-9-tetrahydrocannabinol (triOH-THC).

16. The method of claim 10, wherein the cannabinoid receptor agonist comprises one or more of a naphthoylindole, a naphthylmethylindole, a naphthoylpyrrole, a naphthylmethylindene, a phenylacetylindole and a cyclohexylphenol.

17. The method of claim 10, wherein the hydroxy acid is an alpha hydroxy acid, a beta hydroxy acid or a combination thereof.

18. The method of claim 17, wherein the alpha hydroxy acid is lactic acid, citric acid, glycolic acid, mandelic acid, benzylic acid, malic acid, tartaric acid, gluconolactone, galactonolactone, glucuronolactone, galacturonolactone, gulonolactone, ribonolactone, saccharic acid lactone, pantoyllactone, glucoheptonolactone, mannonolactone, or galactoheptonolactone.

19. The method of claim 17, wherein the beta hydroxy acid is salicylic acid or lipohydroxy acid.

20. The method of claim 10, wherein the composition further comprises a stabilizer selected from the group consisting of guar gum, xanthan gum cellulose hyaluronic acid, polyvinyl pyrrolidone (PVP), alginate, chondritin sulfate, poly gamma glutamic acid, gelatin, chitisin, corn starch and flour, in an amount from about 0.25% to about 30% (w/v).

21. The method of claim 10, wherein the carrier comprises hemp oil and wherein the composition further comprises one or more of a thickening agent, an antibiotic, an antiseptic agent, an antifungal, an antibacterial agent, an analgesic, an antiviral agent or a UV absorbing agent in an amount between 0.1 and 5% by weight of the composition.

22. The method of claim 10, wherein the subject is a human and wherein the composition is topically administered to the subject in an amount between about 100 nmol to about 1 μmol/cm².
